(19) United States
(12) Patent Application Publication
(10) Pub. No.: US 2006/0147569 A1
(43) Pub. Date: Jul. 6, 2006

(54) Anti-depression formulations

(76) Inventors: Hsiang-fu Kung, New Territories (HK); Ling-dong Kong, Nanjing City (CN)

Correspondence Address: KNOBBE MARTENS OLSON & BEAR LLP, 2040 MAIN STREET, FOURTEENTH FLOOR, IRVINE, CA, 92614, US

(21) Appl. No.: 11/029,121

(22) Filed: Jan. 3, 2005

(51) Int. Cl. A61K 36/185; A61K 31/366

(52) U.S. Cl. 424/779; 514/454

(57) ABSTRACT

The present invention relates to the use of Chinese herbs Buguzhi and Fuling extracts in preparation formulations for the treatment of depression and related symptoms.

### FIELD OF INVENTION

The present invention relates to the field of pharmaceutical composition, dietary supplements, health products, and related formulations. In particular, the present invention relates to formulations comprising herbal extracts for the amelioration of depression and related symptoms.

### BACKGROUND OF INVENTION

References which are cited in the present disclosure are not necessarily prior art and therefore their citation does not constitute an admission that such references are prior art in any jurisdiction.

As the competition in the society increases, people are facing more and more pressure, both physically and psychologically, making depression the most common illness for the new century. It is expected that depression will become the world's second serious illness in the year 2020.

Depression is related with malfunctions of the central nervous system, neuroendocrine system and immune system. The current drugs used to treat or control depression have the drawbacks of narrow applications and effective points, having relatively strong toxicity and side effects, discouraging patients to get the drug treatment.

Kong L. D. (2001) reported on in vitro experiments designed to test inhibition of rat brain monoamine oxidase activities by psoralen and isopsoralen and the potential implications for the treatment of affective disorders. Partonen T. (2000) also reported an experiment relating to psoralens for the treatment of seasonal affective disorder. However, the actual effectiveness thereof is not yet proven.

It is thus desirable to develop new formulations to treat depression that do not have the above-mentioned drawbacks and with improved effectiveness.

### SUMMARY OF INVENTION

It is discovered in the present invention that the combination of Psoralea corylifolia L. and that of Poria cocos (Schw.) Wolf. demonstrates unexpected effectiveness in alleviating depression-related symptoms.

In one aspect of the present invention, a pharmaceutical composition comprising the extracts of Psoralea corylifolia L. and Poria cocos (Schw.) Wolf. is provided. In some embodiments, the composition does not contain any of the herbs selected from the group consisting of Radix Ginsen (Ren Shen), Radix Polygoni Multiflori (He Shou Wu), Radix Ophiopogonis (Mai Dong), Radix Codonopsis (Dang Shen), Radix Astragali (Huang Qi) Rhizoma Atractylodis Macrocephalae (Bai Shu), Fructus Ligustri Lucidi (Nv Zhen Zi), Radix Salviae Miltiorrhizae (Dan Shen), Rhizoma Paridis (Chong Lou), Rhizoma Cyperi (Xiang Fu), Rhizoma Atractylodis (Cang Shu), Radix Saposhnikoviae (Fang Feng), Herba Ecliptae (Mo Han Lian), Fructus Tribuli (Ji Li), Radix Lithospermi (Zi Cao), and Radix Glycyrrhizae (Gan Cao).

In an embodiment, the pharmaceutical composition consists essentially of the extract of Psoralea corylifolia L. and the extract of Poria cocos (Schw.) Wolf. In another embodiment, the pharmaceutical composition is consisted of the extract of Psoralea corylifolia L. and the extract of Poria cocos (Schw.) Wolf. In another embodiment, in the pharmaceutical composition, the extract of Psoralea corylifolia L. is Buguzhi extract and the extract from Poria cocos (Schw.) Wolf is Fuling extract. (Buguzhi is the dried seed of Psoralea corylifolia L. Fuling is the dried sclerotium of Poria cocos (Schw.) Wolf.)

In another embodiment, the weight portions of the Buguzhi extract to the Fuling extract range from about 1:1 to about 1:7. In another embodiment, the weight portions of Buguzhi extract to Fuling extract range from about 1:1 to about 1:3. In another embodiment, the weight portions of Buguzhi extract to Fuling extract is about 1:1.5.

In yet another embodiment, the Buguzhi extract of the composition comprises about 95% furocoumarins, wherein said furocoumarins comprises about 7-9% of Psoralen and about 6-8% of Isopsoralen. The Fuling extract of the composition comprises about 57-62% Fuling Polysaccharides.

In another aspect of the present invention, a pharmaceutical composition comprising Furocoumarins and Fuling polysaccharides is provided, wherein said furocoumarins comprising Psoralen and Isopsoralen.

In an embodiment, the proportion of Psoralen, Isopsoralen and Fuling polysaccharides is 7-9:6-8:86-93.

In yet another aspect of the present invention, a method of preparing the pharmaceutical compositions mentioned above is provided. The method comprises the following steps:

a. extracting Buguzhi to form Buguzhi extract,

b. extracting Fuling to form Fuling extract, and

c. combining said Buguzhi extract and said Fuling extract.

In an embodiment, the weight ratio of Buguzhi to Fuling in the method ranges from about 1:2 to about 2:1. In another embodiment, the weight ratio of the Buguzhi to Fuling ranges from about 1:1 to about 2:1. In another embodiment, the weight ratio of Buguzhi to Fuling is about 2:1.

In another embodiment, the size of Buguzhi and Fuling were reduced by grinding, dicing, slicing, or segmentation.

In yet another embodiment, step (b) of the above-mentioned method comprises
i) extracting said Buguzhi with a first extractant to form a first extract solution,
ii) extracting said first extract solution with a second extractant to form a second extract solution comprising residue, and
iii) isolating and drying said residue from said second extract solution.
   wherein one of said extractants is high-polar solvent, the other one of said extractants is low-polar solvent.

In an embodiment, the first extractant is a high-polar solvent and the second extractant is a low-polar solvent. In another embodiment, the first extractant is selected from a group consisting of ethanol, methanol, acetone, and ethyl acetate, and the second extractant is selected from a group consisting of ether, chloroform, dichloromethane. In another embodiment, the first extractant is selected from ethanol, methanol, and acetone. In another embodiment, the first extractant is selected from 55-75% ethanol, 30-95% methanol, and 30-95% acetone and the second extractant is 100% ethyl acetate, 100% ether, 100% chloroform, and 100% dichloromethane. In another embodiment, the first extractant is 55-75% (v/v) ethanol and the second extractant is 100% ethyl acetate.

In yet another embodiment, step (b) of the above-mentioned method further comprises:
i) extracting Fuling with water to obtain a water-soluble extract and a water-insoluble Fuling residue,
ii) extracting the water-insoluble Fuling residue with an alkaline solvent to obtain an alkali-soluble extract,
iii) combining said water-soluble extract and said alkali-soluble extract to form said Fuling extract.

In another embodiment, the step (i) further comprises:
(a) decocting the Fuling in water to form a Fuling solution,
(b) filtering and concentrating said Fuling solution to form a concentrated Fuling solution,
(c) forming precipitation from said concentrated Fuling solution, and
(d) isolating and drying said precipitation to obtain the water-soluble extract.

In another embodiment, the decocting of step (a) was maintained at about 80-100° C. and repeated for three times, 2 hours each time. In another embodiment, the decocting was maintained at about 95-100°.

In another embodiment, the precipitation in step (c) is formed by adding alcohols. In another embodiment, the alcohol is selected from a group consisting of ethanol, methanol, propanol, butyl, and isopropyl. In another embodiment, the alcohol is ethanol. In another embodiment, the alcohol is 95% (v/v). In another embodiment, the final ethanol concentration after addition of said ethanol is about 70% (v/v).

In yet another embodiment, step (ii) comprises:
(a) extracting the Fuling residue with an alkaline solvent, forming an extract solution at pH=11-13,
(b) neutralizing said extract solution with acid, forming neutralized extract solution at pH=6-7,
(c) forming precipitation from said neutralized extract solution,
(d) isolating and drying said precipitate to obtain the alkali-soluble extract.

In another embodiment, the extraction in step (a) comprises soaking the Fuling residue in alkali solvent under room temperature. In another embodiment, the extraction was conducted for three times, each time being 8-12 hour. More preferably, 10 hours.

In another embodiment, the pH value of the extract solution of step (a) is pH 12, and the alkaline solvent is NaOH. In another embodiment, the NaOH was 1M.

In a yet another embodiment, the pH value of the neutralized extract solution of step (b) is 6.5. In another embodiment, the acid is acetic acid. In another embodiment, the acetic acid is 10% (v/v).

In still another embodiment, the alcohol is ethanol. In another embodiment, the final ethanol concentration of step (c) is 60-80% (v/v). In another embodiment, the ethanol concentration is 70% (v/v).

In yet another aspect of the present invention, a method of treatment of symptoms related with depression is provided. The method comprises administration of a therapeutically effective amount of the above-mentioned pharmaceutical compositions. In another embodiment, the effective amount is 100-250 mg/kg/per day. More preferably, 150-200 mg/kg/per day. Most preferably, 200 mg/kg/per day. In another embodiment, the symptoms that can be alleviated by said pharmaceutical compositions comprises anxiety, insomnia, stress, nervousness, emotional disorder, disability to adapt to pressure, despair, failure to adapt to stress, elevated cortisol level, bad appetite, or increased MAO-A and MAO-B activities in the brain.

In still another aspect of the present invention, use of the pharmaceutical compositions mentioned above in the preparation of a medicament for the treatment of depression is provided.

In a further aspect of the present invention, a health supplement or a nutritional supplement comprising herbal extracts is provided. The herbal extracts consisting essentially of Buguzhi extract and Fuling extract. In another embodiment, the herbal extracts consisting of Buguzhi extract and Fuling extract. In another embodiment, the health supplement does not contain any other herbs in addition to Buguzhi and Fuling.

In yet another aspect of the present invention, a formulation for treating depression is provided. The formulation comprises the pharmaceutical composition mentioned above, and the concentration of the composition ranges from 1% to 100% by weight. In another embodiment, the concentration ranges from 10-99%, 20-99%, 30-99%, 40-99%, 50-99%, 60-99%, 70-99%, 80-99%, or 90-99%.

In another embodiment, the ratio of Buguzhi extracts to Fuling extract in the formulation ranges from 1:1 to 1:7. In another embodiment, the ratio ranges from 1:1.5 to 1:6. In another embodiment, the ratio ranges from 1:1.5-1:3. In another embodiment, the ratio of Buguzhi extracts to Fuling extract is 1:1.5.

Further aspects of the present invention are described in the following numbered paragraphs.

1. A pharmaceutical composition comprising the extract of Psoralea corylifolia L. and extract of Poria cocos (Schw.) Wolf., wherein said composition is in the absence of the herbs selected from the group consisting of Radix Ginsen, Radix Polygoni Multiflori, Radix Ophiopogonis, Radix Codonopsis, Radix Astragali, Rhizoma Atractylodis Macrocephalae, Fructus Ligustri Lucidi, Radix Salviae Miltiorrhizae, Rhizoma Paridis, Rhizoma Cyperi, Rhizoma Atractylodis, Radix Saposhnikoviae, Herba Ecliptae, Fructus Tribuli, Radix Lithospermi, and Radix Glycyrrhizae.

2. A pharmaceutical composition according to paragraph 1, consisting essentially of the extract of Psoralea corylifolia L. and the extract of Poria cocos (Schw.) Wolf.

3. A pharmaceutical composition according to paragraph 1, consisting of the extract of Psoralea corylifolia L. and the extract of Poria cocos (Schw.) Wolf.

4. The pharmaceutical composition according to paragraph 1, wherein said extract of Psoralea corylifolia L. is Buguzhi extract and said extract from Poria cocos (Schw.) Wolf. is Fuling extract.

5. The pharmaceutical composition according to paragraph 4, wherein the weight portion of Buguzhi extract and Fuling extract ranges from about 1:1 to about 1:7.

6. The pharmaceutical composition according to paragraph 4, wherein the portion of Buguzhi extract and Fuling extract ranges from about 1:1 to about 1:3.

7. The pharmaceutical composition according to paragraph 4, wherein the portions of Buguzhi extract and Fuling extract is about 1:1.5.

8. The pharmaceutical composition according to paragraph 4, wherein the Buguzhi extract comprises about 95% furocoumarins, wherein said furocoumarins comprising about 7-9% of Psoralen and about 6-8% of Isopsoralen.

9. The pharmaceutical composition according to paragraph 4, wherein the Fuling extract comprises about 57-62% Fuling Polysaccharides.

10. A pharmaceutical composition comprising furocoumarins and Fuling polysaccharides, wherein said furocoumarins comprising Psoralen and Isopsoralen.

11. The pharmaceutical composition according to paragraph 10, wherein the proportions of psoralen, isopsoralen and Fuling polysaccharides in the composition is 7-9:6-8:86-93.

12. A method of preparing the pharmaceutical composition of paragraph 4, comprising:
a. extracting Buguzhi to form Buguzhi extract;
b. extracting Fuling to form Fuling extract, and
c. combining said Buguzhi extract and said Fuling extract.

13. The method according to paragraph 12, wherein the weight ratio of Buguzhi to Fuling ranges from about 1:2 to about 2:1.

14. The method according to paragraph 12, wherein the weight portion of Buguzhi to Fuling ranges from about 1:1 to about 2:1.

15. The method according to paragraph 12, wherein the weight portion of Buguzhi to Fuling is about 2:1.

16. The method according to paragraph 12, wherein said extracting of Buguzhi in step a. further comprises:
i. extracting said Buguzhi with a first extractant to form a first extract solution;
ii. extracting said first extract solution with a second extractant to form a second extract solution comprising residue; and
iii. isolating and drying said residue from said second extract solution;

wherein one of said extractants is a high-polar solvent, the other of the extractants is a low-polar solvent.

17. The method according to paragraph 16, wherein said first extractant is selected from the group consisting of ethanol, methanol, and acetone.

18. The method according to paragraph 16, wherein said first extractant is 65% (v/v) ethanol.

19. The method according to paragraph 16, wherein said second extractant is selected from the group consisting of ethyl acetate, ether, chloroform, and dichloromethane.

20. The method according to paragraph 16, wherein said second extractant is 100% (v/v) ethyl acetate.

21. The method according to paragraph 12, wherein the extracting of Fuling in step b. further comprises
i. extracting Fuling with water to obtain a water-soluble extract and a water-insoluble Fuling residue;
ii. extracting the water-insoluble Fuling residue with an alkaline solvent to obtain an alkali-soluble extract; and
iii. combining said water-soluble extract and alkali-soluble extract to form said Fuling extract.

22. The method according to paragraph 21, wherein step (i) further comprises
a. decocting the Fuling in water to form a Fuling solution;
b. filtering and concentrating said Fuling solution to form a concentrated Fuling solution;
c. precipitating said concentrated Fuling solution to form precipitation; and
d. isolating and drying said precipitation to obtain the water-soluble extract.

23. The method according to paragraph 22, wherein said precipitation in step (c) is formed by adding alcohol.

24. The method according to paragraph 23, wherein said alcohol is 95% (v/v) ethanol.

25. The method according to paragraph 21, wherein step (ii) further comprises:
a. extracting the water-insoluble Fuling residue with an alkaline solvent to form an extract solution, wherein said extract solution having a pH value of 11-13;
b. neutralizing said extract solution with acid to pH=6-7 to form a neutralized extract solution;
c. precipitating said neutralized extract solution to form precipitation; and
d. isolating and drying said precipitate to obtain the alkali-soluble extract.

26. The method according to paragraph 25, wherein said alkaline solvent is 1M NaOH.

27. The method according to paragraph 25, wherein said precipitation is formed by adding alcohol.

28. The method according to paragraph 27, wherein said alcohol is 95% (v/v) ethanol.

29. A method of treatment of depression, comprising administering of a therapeutically effective amount of the pharmaceutical composition of paragraph 1.

30. The method according to paragraph 29, wherein the effective amount is 100-250 mg/kg/d.

31. The method according to paragraph 29, wherein said treatment comprises modulating at least one of the following symptoms: anxiety, insomnia, stress, nervousness, emotional disorder, disability to adapt to pressure, despair, failure to adapt to stress, enhanced cortisol level, and enhanced MAO-A and MAO-B activities in the brain, reduced energy, chronic fatigue syndrome, postpartum depression, anxious depression, atypical depression, anxious mood, irritable mood, and melancholic and atypical symptom, violent acts, suicidal behavior or suicidal ideation, sleep disturbance, decrease in appetite, eating disorder, concentration, psychomotor agitation/retardation.

32. A health supplement comprising the pharmaceutical composition of paragraph 1.

33. A nutritional supplement comprising the pharmaceutical composition of paragraph 1.

34. A formulation for treating depression comprising composition of paragraph 4, wherein the concentration of said composition ranges from 1% to 100% by weight.

35. The formulation according to paragraph 34, wherein the ratio of Buguzhi extracts to Fuling extract ranges from 1:1 to 1:7.

36. The formulation according to paragraph 34, wherein the ratio of Buguzhi extracts to Fuling extract ranges from 1:1.5 to 1:6.

37. The formulation according to paragraph 34, wherein the ratio of Buguzhi extracts to Fuling extract is from 1:1.5 to 1:3.

38. Use of the pharmaceutical composition of paragraph I in the preparation of a medicament for the treatment of depression.

### BRIEF DESCRIPTION OF THE DRAWINGS

FIG. 1 shows a process of making the Fuling extract according to one aspect of the present invention.

FIG. 2 shows the HPLC chromatogram of standard Psoralen and Isopsoralen.

FIG. 3 shows the HPLC chromatogram of the Buguzhi extract produced according to one aspect of the present invention.

### DETAILED DESCRIPTION

As used in the present specification and claims, the term “comprising” “comprise,” “comprises,” and “comprising” mean “including, but not necessarily limited to”. For example, a method, apparatus, molecule or other item which contains A, B, and C may be accurately said to comprise A and B. Likewise, a method, apparatus, molecule or other item which “comprises A and B” may include any number of additional steps, components, atoms or other items as well. The term “consisting of”, “consisted of”, “consists of” mean that the product (or method) has the recited elements (or steps) and no more. The term “consisting essentially of”, “consisted essentially of”, and “consists essentially of” mean that in addition to the recited elements, the product (or method) necessarily includes the listed elements and is open to unlisted elements that do not materially affect the basic and novel properties of the product (or method). Particularly, “consisting essentially of” excludes other elements from having any essential significance to the combination, that is, it allows some “reading on” additional unspecified substances, i.e., those which do not materially affect the basic and novel characteristics of the claimed invention.

As used herein, “Buguzhi” and “Fuling” refer to the traditional Chinese herbs defined below. For ease of references, the naming of the two herbs is based on the official pronunciation system of the People's Republic of China.

Buguzhi, also known as Fructus Psoraleae, Psoralea Fruit, Malaysian Scurf-pea, is the dried ripe seed of Psoralea corylifolia L. Buguzhi is available commercially in various forms, such as the original dried fruit, powder, or piece. It is usually taken orally after being dried. The dried Fructus Psoraleae can be taken raw or sauteed. For detailed information, please visit the following URL: http://www.e2121 .com/herb_db/viewherb.php3?viewid=576&setlang=1

As used herein, the term “extract”, “herbal extract”, and “herbal extracts” refer to a concentrated preparation of a plant obtained by extracting the active constituents therefrom using the general methods recited herein and other equivalent methods generally known in the art. The term “water-soluble extract,” as used herein, means a plant extract which is soluble in water; the term “alkali-soluble extract” means a plant extract which is soluble in alkaline solvent.

As used herein, “Buguzhi extract” most generally refers to the composition isolated from the dried seeds or other parts of the plant of the Psoralea family according to a specified extraction procedure, and preferably refers to the composition isolated from the seeds of Psoralea corylifolia L. according to a specified extraction procedure. These extracts comprise Psoralen, Isopsoralen, and furocoumarins.

Fuling as used herein refers to the dried sclerotium of Poria cocos (Schw.) Wolf., also known as Tuckahoe or Bokryun or Poria or hoelen. Fuling is available commercially in various forms, such as the original dried fungi, powder, or slice. For detailed information, please visit the following URL: http://www.e2121.com/herb_db/viewherb.php3?viewid=239&setlang=1

As used herein, “Fuling extract” most generally refers to the composition isolated from the dried scierotium, mycelia or other parts of the fungus of the Poria family, according to a specified extraction procedure, and preferably refers to the composition isolated from dried sclerotium of Poria cocos (Schw.) Wolf. according to a specified extraction procedure. These extracts comprise polysaccharides, said polysaccharides comprise pachymaran, glucan, pachymose, and pachyman etc. As used herein, the term “Fuling polysaccharides” refers to the polysaccharides contained in Fuling extract.

As used herein, the term “high-polar solvent” refers to an organic polar solvent which Snyder polarity index is above 5, a “low-polar solvent” refers to an organic solvent which Snyder polarity index of said solvent is below 5.

Antidepressant Effects

In order to investigate the antidepressant effects of the pharmaceutical composition, tail suspension test (TST), forced swimming test (FST) in mice, and chronic mild stress (CMS) model in rats were conducted. The significance of these models are explained below.

TST

A major symptom of depression is decrease of motivational behaviors. In the TST model, the tail-suspended mice struggle in attempt to overcome the abnormal body positions, which is a motivational behavior. After a period of time, the mice started to show occasional immobility, representing a “despair” mental state. The immobility is regarded as “failure to adapt to stress” and can be served as an indicator of the level of depression. The effectiveness of anti-depression drugs could be derived by measuring the duration of immobility after the mice had been tail-suspended for a period of time. The model is sensitive to anti-depression drugs and is widely adopted as a means to screen such drugs. (Steru L, Chermat R, Thierry B, Simon P. 1985.)

FST

A major symptom of depression is decrease of motivational behaviors. In the FST model, the mice were forced to swim in a restricted space. First, they struggle in attempt to escape, which is a motivational behavior. After a period of time, the mice started to show occasional immobility, representing a “despair” mental state. The immobility is regarded as “failure to adapt to stress” and can be served as an indicator of the level of depression. The effectiveness of anti-depression drugs could be derived by measuring the duration of immobility after the mice had been forced to swim for a period of time. The model is sensitive to anti-depression drugs and is widely adopted as a means to screen such drugs. (Porsolt et al., 1977)

Chronic Mild Stress (CMS) Model

In the CMS model, the recovery of sucrose intake, inhibition of brain monoamine oxidase A and B (MAO-A and MAO-B) activities, and reduction in cortisol level in CMS-treated rats are used as indicators of effectiveness of anti-depression drugs.

In the CMS model, rats are exposed sequentially, over a period of weeks, to a variety of mild stressors, and the measure most commonly used to track the effects is a decrease in consumption of a palatable sweet solution. This counteracts the usual effect of antidepressants to increase sucrose intake in CMS model of rats. Further studies have shown that rats subjected to CMS exhibit biochemical and physiological impairments. These impairments could be reversed by chronic administration of antidepressants such as tricyclic antidepressants, selective serotonin reuptake inhibitors and monoamine oxidase inhibitors. On the other hand, non-antidepressant drugs were found to be ineffective in the CMS model. The model has good predictive validity (behavioural changes are reversed by chronic treatment with a wide variety of antidepressants), face validity (almost all demonstrable symptoms of depression have been demonstrated), and construct validity (CMS causes a generalized decrease in responsiveness to rewards, comparable to anhedonia, the core symptom of the melancholic subtype of major depressive disorder). (Willner P. 1997)

When animals are stimulated by CMS, their sucrose intake will decrease. On the other hand, their MAO-A and MAO-B activities in brain and the serum cortisol level will increase. Agents capable of recovering the sucrose intake, inhibiting the MAO-A or MAO-B activities, or lowering the cortisol level in the CMS-treated animals are believed to be effective anti-depression drugs.

### EXAMPLE 1

### Preparing the Buguzhi Extract

Reagent and apparatus:
The seeds of P. corylifolia (Buguzhi): purchased from the Herbal Medicine Co-operative Association of Jiangsu Province, China, in May 2001, and identified by the biology department of Life Sciences, Nanjing University, P. R. China. A voucher specimen (No. NJ-34060) was deposited at the Herbarium of the School of Life Sciences, Nanjing University, Nanjing 210093, P. R. China.
95% (v/v) Ethanol: purchased from Nanjing Chemical Reagent No.1 Factory, P. R. China, product code: 1370400201.
65% Ethanol (v/v) preparation: 684 ml of 95% Ethanol was added to 316 ml water.
100% Ethyl acetate: purchased Nanjing Chemical Reagent No.1 Factory, P. R. China, product code: 1370401001.
Percolator (1000 ml): purchased from Tianjin You Feng Technology Glass Co., Ltd., P. R. China, product code: 119-02-10.
Separatory funnel (1000 ml): purchased from Nanjing Sanlai Laboratory Glassware Company, P. R. China, product code: 8543.

The air-dried seeds of P. corylifolia (100 g) were powdered, mixed with 75 ml of 65% ethanol and stayed soaked for 2 hours before placing the mixture into the percolator. 200 ml of 65% ethanol was added to the mixture in the percolator and allowed to stay for 24 hours before adding 1300 ml of 65% ethanol to the percolator. 1500.0 mL of filtrate solution was collected at a rate of 1.5 mL/min. The filtrate was concentrated under reduced pressure at 60° C. to remove ethanol in a rotavapor apparatus (Buchi Labortechnik AG; Switzerland), obtaining 250.0 mL mixture liquid. The mixture liquid was extracted for 8 times, each time using 250 ml of 100% ethyl acetate. The ethyl acetate phase was collected, and the solvent was evaporated under reduced pressure at 60° C. until most of ethyl acetate had been removed. The residue from the ethyl acetate extraction was dried under reduced pressure at 60° C., obtaining 12.10 g of Buguzhi extract (yield 12.10%).

### EXAMPLE 2

### Preparing the Fuling Extract

Reagent and Apparatus
The dried sclerotium of Poria cocos (Fuling): purchased from the Herbal Medicine Co-operative Association of Jiangsu Province, China, in May 2001, and identified by the biology department of Life Sciences, Nanjing University, P. R. China. A voucher specimen (No. NU-62004) was deposited at the Herbarium of the School of Life Sciences, Nanjing University, Nanjing 210093, P. R. China.
95% Ethanol: purchased from Nanjing Chemical Reagent No. 1 Factory, P. R. China, product code: 1370400201.
Sodium hydroxide: purchased from Nanjing Chemical Reagent No. 1 Factory, P. R. China, product code: 1370500701. 1 mol/L of NaOH was prepared by adding 40 g of Sodium hydroxide to 1000 ml water.
Acetic acid (99% v/v): purchased from Nanjing Chemical Reagent No. 1 Factory, P. R. China, product code: 1370401201. 10% of acetic acid was prepared by adding 100 ml acetic acid to 900 ml water.
Beaker (2000 ml): purchased from Shanghai Shen Bo Glass Instrument Co., P. R. China, product code: SF-1101-15.
Beaker (5000 ml): purchased from Shanghai Shen Bo Glass Instrument Co. purchased from Shanghai Shen Bo Glass Instrument Co., P. R. China, product code: SF-1101-17.
Separatory funnel (1000 ml): purchased from Nanjing San'ai Laboratory Glassware Company, P. R. China, product code:8543.
Flask (2000 ml): purchased from Nanjing San'ai Laboratory Glassware Company P. R. China, product code: 1117.
Spheroidal condensation tube: purchased from Beijing Glass Instruments Factory P. R. China, product code: yq230503.
Percolator (1000 ml): purchased from Tianjin You Feng Technology Glass Co., Ltd. P. R. China, product code: 119-02-10.
RE52-1 Rotary Evaporator: purchased from Shanghai Hu Xi Analysis Instrument Factory Co., Ltd, P. R. China.
HY30-01 Electric vacuum drying oven: purchased from Taijiang Medical Apparatus and Instruments Factory of Fuzhou City, P. R. China.
HH-S Electric-heated thermostatic water bath: purchased from Jiangsu Dongtai Electrical Apparatus Works, P. R. China.

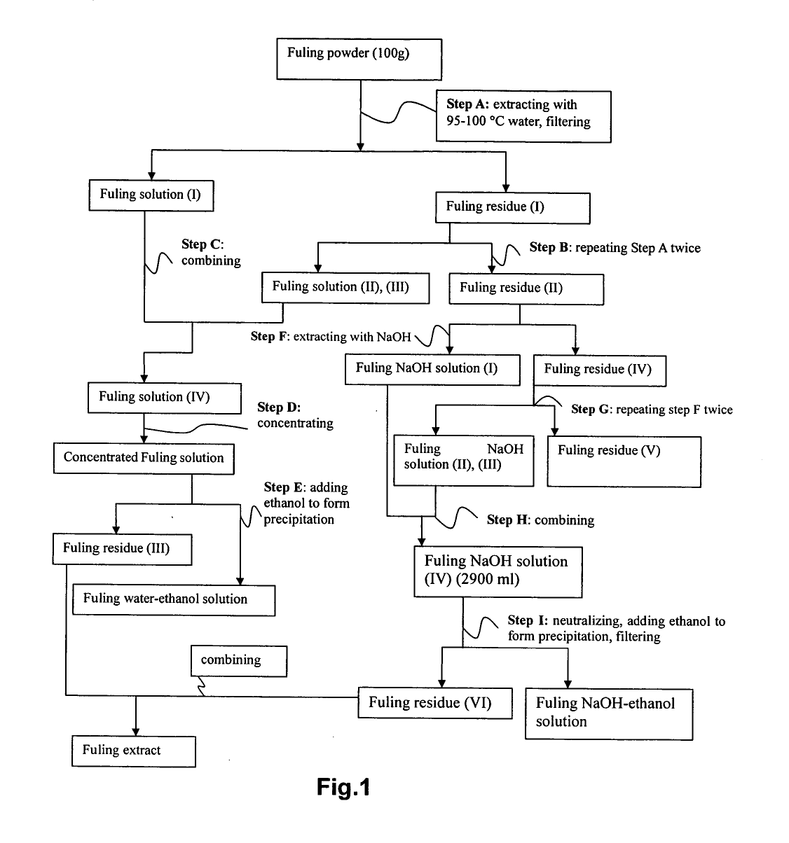

Referring to FIG. 1, the sclerotium of Poria cocos (Fuling, 100 g) was powdered and extracted with 1000 ml of distilled water at 95-100° C. for 2 hours before filtering in step A, forming a Fuling solution (I) (the filtrate) and a Fuling residue (I). In step B, The Fuling residue (I) was extracted for another 2 times with the same procedures in step A, forming a Fuling solution (II), and (III) and Fuling residue (II), The Fuling solutions (I), (II), (III) were combined in step C, forming a Fuling solution (IV). The Fuling solution (IV) was condensed by a rotor evaporator in step D, forming 300 ml of concentrated Fuling solution. In step E, the concentrated Fuling extract was added with 840 ml of 95% ethanol while stirring to a final concentration of 70% ethanol (v/v) and was kept still overnight at 4° C. to allow precipitation. The precipitate was recovered by filtration through filter paper, and freeze-dried, forming a total of 1.23 g of Fuling residue (III) in dried powder form and Fuling water-ethanol solution. In step F, The Fuling residue (II) was soaked in 1000 ml of 1M NaOH for 10 hours under room temperature before filtering, forming Fuling NaOH solution (I) (the filtrate) and Fuling residue (IV). In step G, the Fuling residue (IV) was extracted for another two times with the same procedures in step F, forming the Fuling residue (V) and Fuling NaOH solutions (II) and (III). In step H, the Fuling NaOH solutions (I), (II), (III) were combined to form 2900 ml of Fuling NaOH solution (IV). In step I, the Fuling NaOH solution (IV) was neutralized with 10% of acetic acid to pH=6, followed with adding 8120 ml of 95% ethanol while stirring and kept overnight at 4° C. to form precipitation. The precipitate was recovered by filtration through filter paper, and freeze-dried, forming a total of 35.69 g of Fuling residue (VI) n dried powder form and Fuling NaOH-ethanol solution.

The Fuling residues (III) and (VI) were combined to yield 36.92 g of Fuling extract (yield 36.92%).

### EXAMPLE 3

### Methods Used to Analyze the Components of Buguzhi Extract

The components (psoralen and isopsoralen) of the Buguzhi extract produced in Example 1 were analyzed by HPLC, using ultraviolet as detection, which presented lower quantification and detection limits. This method showed good accuracy, precision, and linearity.

Reagents

Spectroscopy-grade acetonitrile and methanol were purchased from International Laboratory (USA). Water was purified using a Milli-Q system (Millipore). Psoralen and isopsoralen standards were supplied by Chinese National Institute for the Control of Pharmaceutical and Biological Products. Stock mixtures of these standards were made up from the individual solutions in methanol and used as external standards.

Apparatus and Chromatography Conditions

The analysis was performed on Agilent 1100 liquid chromatography system with DAD detector. HPLC separation of the psoralens and isopsoralens was performed using a Zorbax XDB RP-C 18 column (4.6 mm i.d.×25 cm long and 5 m particle diameter) together with a RP-C18 guard column (4.6 mm i.d.×1.25 cm long) used to protect the analytical column. Before use, the solvents were filtered through a 0.45-μm HV filter (Millipore) then degassed for 20 min in an ultrasonic bath. Elution was performed with acetronitrile-water (40:60, v/v) at a flow-rate of 1.0 ml min-1. 1.0 μl were subjected to quantitative analysis and detection of the peaks was recording at 246 nm. All chromatography analysis were performed at 22° C.

Sample Preparation

15 mg of the Buguzhi extract was dissolved in methanol on a volumetric flask (25 ml) and filtered through 0.45 μm Millex filter in order to be analysed by HPLC.

Results

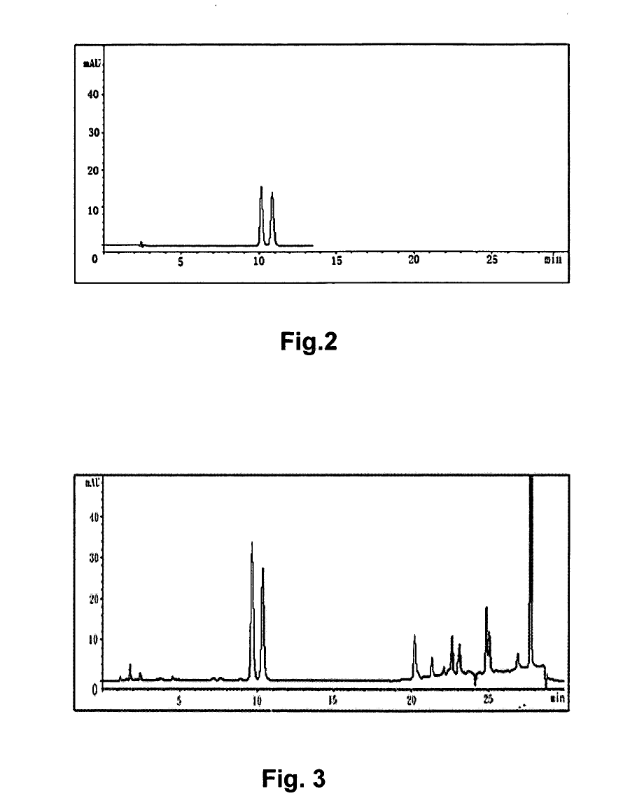

HPLC analysis showed baseline separation for the compounds of interest, which could be analyzed in a satisfactory time interval of less than 20 minutes (psoralen 9.90 min and isopsoralen 10.60 min). The chromatogram of standard psoralen and isopsoralen by HPLC is shown in FIG. 2. In the time intervals, where the compounds eluted, were free of interference in the Buguzhi extract tested employed sample preparation optimized. The chromatogram of the Buguzhi extract by HPLC is shown in FIG. 3. The identification of psoralen and isopsoralen in the Buguzhi extract were performed by comparison of their retention time with the authentic standards and standards addiction in the samples.

The calibration curves were determined by linear regression. The calibration curve for psoralen was linear in the range of 0.00219-0.3501 μg and 0.00206-0.3301 μg for isopsoralen.

Linear regression formula for psoralen: y=7289.9-14.593 x, r=0.9998.

Linear regression formula for isopsoralen: y=7214.4-12.456 x, r=0.9997

where y=peak areas ratio, x=quality (μg), r=determination coefficiente.

The linearity of the method was determined by linear regression. Detection limits were 0.0003 μg for psoralen and 0.0003 μg for isopsoralen. The quantification limits by were 0.0011 μg for psoralen and 0.0010 μg for isopsoralen.

Analysis of the Buguzhi Extract

Five batches of Buguzhi extracts were produced following the procedures in Example 1. TLC calibration and colorimetry were conducted to determine the content of the total furocoumarins of the Buguzhi extract samples. The content thereof is about 95%.

The components contained in the furocoumarins of the Buguzhi extract were analyzed and summarized in Table 1, which shows the contents of psoralen and isopsoralen in the Buguzhi extract analyzed by HPLC. Five independent analyses were performed for each sample. The Contents (%) (mean±S.D.) of psoralen and isopsoralen in the five batches of Buguzhi extract employing the HPLC method were summarized as Table 1.

TABLE 1

| The Buguzhi extract | Psoralen (%) | Isopsoralen (%) |
|---|---|---|
| 1 | 8.41 ± 0.12 | 7.18 ± 0.10 |
| 2 | 8.36 ± 0.11 | 7.68 ± 0.06 |
| 3 | 7.79 ± 0.13 | 6.52 ± 0.08 |
| 4 | 7.53 ± 0.09 | 6.26 ± 0.12 |
| 5 | 7.96 ± 0.11 | 6.66 ± 0.06 |

S.D., standard deviations; S.D. of five determinations.

The average amount of Psoralen in Buguzhi extract produced according to Example 1, according to the above data, is 8.01%, and that of Isopsoralen is 6.86%.

### EXAMPLE 3.1

### Methods Used to Analyze the Components of Fuling Extract

The components of Fuling extract (polysaccharids) were analyzed by phenol-sulfuric acid method. The phenol-sulfuric method (Dubosis et al., 1956) is a calorimetric reaction frequently used to determine simple sugars, oligosaccharides, polysaccharides and their derivatives due to its simplicity and economy of its reagents.

Reagents

Sulfuric acid, reagent grade 95.5% was purchased from Sigma-Aldrich (St. Louis, Mo., USA), product code: 32,050-1.
Reagent grade Phenol was purchased from Sigma-Aldrich (St. Louis, Mo., USA), product code: P 4161.
D-Glucose was obtained from from Sigma-Aldrich (St. Louis, Mo., USA), product code: G 5250.
5 g/dl phenol-water solution: prepared by adding 100 ml of distilled water to 5 g of redistilled phenol.
10 g/dl glucose-water solution: prepared by adding 100 ml of distilled water to 10 g of glucose.
   Apparatus

U-3000 Spectrophotometer: Hitachi, Ltd. Tokyo, Japan.

Sample Preparation

Fuling extract 5 mg was dissolved in water on a volumetric flask (100 ml).

Phenol-sulfuric Acid Method

The glucose was used as the standard for the calibration curve. The basic protocol of Dubosis et al. (1956) was followed. Briefly, the 0.2, 0.4, 0.6, 0.8, 1 and 1.2 ml of 10 g/dl glucose is pipetted into a calorimetric tube, respectively, then adding the distilled water to final volume 2 ml (containing 20-120 mg glucose). 1 ml of 5% (w/v) phenol is added. Then 5 ml of concentrated sulfuric acid is added rapidly, the stream of acid being directed against the liquid surface rather than against the side of the test tube in order to obtain good mixing. The tubes are allowed to stand 10 minutes, then they are shaken and placed for 15 minutes in a water both at 25 to 30° C. before readings are taken at 490 nm. Blanks are prepared by substituting distilled water for the sugar solution.

Linear least squares regression of the absorbance as a function of the concentrations was performed to determine correlation coefficients. The equation parameters (slop and intercept) of the standard curve were used to obtain concentration values for quality control and samples of topic solutions.

Linear Regression

Formula: y=0.0068+6.6929x, r=0.9994

where y=absorbance, x=glucose (mg), r=determination coefficient

Determination of Polysaccharides Content in Fuling Extract

The polysaccharides in Fuling extract were quantified by phenol-sulfuric acid method using various levels (20-120 mg) of D-glucose to construct the calibration curve. Color developed at 490 nm was monitored by a spectrophotometer. Five separate experiments were each tested in quinary.

Briefly, the Fuling extract (2 ml) was pipetted into a colorimetric tube, respectively. 1 ml of 5% (w/v) phenol was added. Then 5 ml of concentrated sulfuric acid was added rapidly, the stream of acid being directed against the liquid surface rather than against the side of the test tube in order to obtain good mixing. The tubes were allowed to stand 10 minutes, then they were shaken and placed for 15 minutes in a water both at 25 to 30° C. before readings were taken at 490 nm. Blanks were prepared by substituting distilled water for the extract solution. The amount of sugar was then determined by reference to a standard curve previously constructed for the glucose under examination. Data points were taken in quinary.

Analysis of the Fuling Extract

The amount of polysaccharides (%) in Fuling extract in five Fuling extract samples were analyzed according to the above-described methods and summarized in Table 2. A total of five batches of Fuling extracts were produced following the procedures in Example 2. The components therein were analyzed and summarized in Table 2.

TABLE 2

| Fuling extract | Polysaccharides (%) |
|---|---|
| 1 | 60.31 ± 1.39 |
| 2 | 58.36 ± 1.11 |
| 3 | 59.19 ± 1.48 |
| 4 | 58.56 ± 1.06 |
| 5 | 59.95 ± 1.19 |

### EXAMPLE 4

### Preparing Formulations

In the following experiments, the following compositions were prepared:
Composition I: Buguzhi extract: Fuling extract=1:1.5, corresponding to Buguzhi: Fuling=2:1.
Composition II: Buguzhi extract: Fuling extract=1:3, corresponding to Buguzhi: Fuling=1:1.
Composition III: Buguzhi extract: Fuling extract=1:6, corresponding to Buguzhi: Fuling=1:2.

The compositions so obtained were then used for the formulation of a preparation with the required concentration using conventional pharmaceutically accepted additives suitable for oral or parenternal administration to CMS treated rats.

### EXPERIMENT 1

### TST and FST Tests on Mice

Materials

1. Animals

Male ICR mice (26±2 g) were used. The mice were housed in polycarbonate cages. They were maintained on a 12-h alternating light-dark schedule in a temperature-controlled (25±2° C.) animal room. Mice were given free access to food and tap water. They were handled for at least 7 days prior to the administration. All animal procedures were approved by the Nanjing University Animal Welfare Committee and conducted in accordance with the guideline of the China Council on Animal Care.

2. Main Reagents

Buguzhi Extract was prepared according to procedures described in Example 1.
Fuling Extract was prepared according to procedures described in Example 2.
Compositions I: Buguzhi extract: Fuling extract=1:1.5.
Composition II: Buguzhi extract: Fuling extract=1:3.
Composition III: Buguzhi extract: Fuling extract=1:6.
Fluoxetine Hydrochloride: purchased from Sigma-Aldrich (St. Louis, Mo., USA), product code: F132.
Saline: (used for negative control): purchased from Sigma-Aldrich (St. Louis, Mo., USA), product code 150-3.
   3. Administration and Doses

Each group of mice received administration at doses listed below:
Saline group (negative control): 5 ml/kg/day.
Buguzhi extract group: 80 mg/kg/day, 50 mg/kg/day, 28.5 mg/kg/day of the Buguzhi extract.
Fuling extract group: 171.5 mg/kg/day, 150 mg/kg/day, 120 mg/kg/day of Fuling extract.
Composition I group: 200 mg/kg/day of Composition I.
Composition II group: 200 mg/kg/day of Composition II.
Composition III group: 200 mg/kg/day of Composition III.
Fluxetine group (positive control): 26 mg/kg/day.

The animals were deprived of food but not water for 1.5 h prior to drug administration of. Fuling extract, Composition I, II, III and fluoxetine at various concentrations were dissolved in saline. The volume of the suspension administered was based on body weight measured immediately prior to each dose, respectively. All drugs were given orally once daily at 13:00-14:00.

As showed in Table 3 and Table 4, the two negative control groups were administered with saline for 3 and 7 days, respectively. The other twenty groups received the Buguzhi extract at 80, 50 and 28.5 mg/kg, Fuling extract at 120, 150 and 171.5 mg/kg, Composition I, II and III at 200 mg/kg, and fluoxetine at 26 mg/kg for 3 and 7, respectively. No abnormal reaction was observed in animals under these doses in each group. The behavioral tests were conducted 1 h after the last treatment, respectively.

Method

1. Tail Suspension Test (TST) in Mice

The tail suspension test was based on the method of Steru (Steru, et al., 1985). Briefly, mouse was individually suspended by the tail with clamp (1 cm distant from the end) for 6 min in a box (30×30×25 cm) with the head 5 cm to the bottom. Testing was carried out in a darkened room with minimal background noise. The duration of immobility time was recorded during the final 4 min interval of the test. The animals were tested used only once in this test.

2. Forced Swimming Test (FST) in Mice

The studies were carried out on mice according to the method of Porsolt (Porsolt, et al., 1977). Briefly, mouse was individually forced to swim individually for 6 min, in glass cylinders (18 cm in height; 14 cm in diameter), containing 25±1° C. water 10-cm deep, so mice could not support themselves by touching the bottom with their feet. The duration of immobility time was measured during the final 4 min interval of the test. At the end of each swimming test, the mice were paper towels dried, placed in heated cages for 15 min, and than returned to their housing condition. The animals were tested used only once in this test.

3. Statistical Analysis

All data were expressed as mean±SEM, and analyzed using one-way analysis of variance (ANOVA). A value of p<0.05 was considered to be statistically significant in all the cases. The calculation of P values was performed by employing the Bonferroni alpha correction to avoid false positive results.

Results

Table 3 shows the effects of the doses on the duration of immobility in the TST in mice. (Mean±S.E.M., n=12). Table 2 shows the effects of the doses on the duration of immobility in the FST in mice (Mean±S.E.M., n=12).

TABLE 3

| | | Duration of immobility(s) | |
|---|---|---|---|
| | | (days) | |
| Group | Dose (mg/kg) | 3 | 7 |
| Negative control | — | 108.9 ± 13.7 | 112.4 ± 10.9 |
| Buguzhi extract | 80 | 67.4 ± 8.6* | 64.8 ± 7.5* |
| | 50 | 78.5 ± 7.1* | 74.0 ± 5.9* |
| | 28.5 | 85.5 ± 6.5 | 90.2 ± 6.2 |
| Fuling extract | 171.5 | 76.3 ± 6.9* | 68.4 ± 6.0* |
| | 150 | 79.6 ± 5.6 | 88.3 ± 9.6 |
| | 120 | 96.3 ± 8.6 | 95.4 ± 7.1 |
| Composition I | 200 | 55.2 ± 8.6** | 49.1 ± 4.6*** |
| Composition II | 200 | 58.8 ± 5.9** | 64.0 ± 10.9* |
| Composition III | 200 | 71.2 ± 7.5* | 62.2 ± 5.0* |
| Fluoxetine | 26 | 23.2 ± 5.2* | 28.8 ± 6.4* |
| (positive control) | | | |

*P < 0.05,
**P < 0.01,
***P < 0.001 when compared with control groups.

TABLE 4

| | | Duration of immobility(s) (days) | |
|---|---|---|---|
| Group | Dose (mg/kg) | 3 | 7 |
| Negative control | — | 116.8 ± 11.7 | 121.8 ± 10.9 |
| Buguzhi extract | 80 | 66.5 ± 8.2** | 74.6 ± 8.6** |
| | 50 | 83.3 ± 6.5* | 76.6 ± 10.1* |
| | 28.5 | 89.2 ± 8.6 | 85.5 ± 6.5 |
| Fuling extract | 171.5 | 72.3 ± 7.9* | 75.4 ± 6.0* |
| | 150 | 87.3 ± 6.6 | 84.7 ± 6.3 |
| | 120 | 104.4 ± 10.2 | 96.7 ± 8.5 |
| Composition I | 200 | 59.4 ± 11.2** | 64.3 ± 9.5*** |
| Composition II | 200 | 75.3 ± 6.8* | 74.0 ± 9.6** |
| Composition III | 200 | 80.4 ± 6.8* | 76.3 ± 10.5* |
| Fluoxetine | 26 | 67.6 ± 10.2* | 67.1 ± 8.9* |
| (positive control) | | | |

*P < 0.05,
**P < 0.01,
***P < 0.001 when compared with control groups.

Conclusion

1. The compositions of Buguzhi and Fuling extracts at proper ratios and doses are effective in reducing the duration of immobility of the TST and FST mice.

2. The effectiveness of the compositions from the strongest to the weakest: Composition I>Composition II>Composition III.

### EXPERIMENT 2

### Behavioral and Biochemical Studies in Chronic Mild Stress (CMS) Model of Depression in Rats

Materials

1. Animals

Male Wistar rats, weighing 180-220 g at the start of the experiment, were used in the experiments. During the one week before experiment, each rat was housed individually in a home cage, and had free access to food and water. They were maintained on a 12 h light/dark cycle under controlled temperature 25±2° C. and tested in the light phase.

The animals were cared in accordance with the principles and guidelines of the Guide for the Care and Use of Laboratory Animals, China Council on Animal Care. All animal procedures were approved by the Nanjing University Animal Welfare Committee.

2. Main Reagents
Composition I: Same as used in Experiment 1, i.e., Buguzhi extract: Fuling extract=1:1.5.
5-Hydroxytrptamine (5-HT) (used as specific substrates for MAO-A): purchased from Sigma-Aldrich (St. Louis, Mo., USA), product code: H 9523.
β-phenylethylamine (PEA) (used as specific substrates for MAO-B): purchased from Sigma-Aldrich (St. Louis, Mo., USA), product code: P 2641.
Fluoxetine Hydrochloride: purchased from Sigma-Aldrich (St. Louis, Mo., USA), product code: F132.
Saline: (used for negative control): purchased from Sigma-Aldrich (St. Louis, Mo., USA), product code 150-3.
Sucrose: purchased from Sigma-Aldrich (St. Louis, Mo., USA), product code: S 7903. Sucrose solution 1% (w/v): 10 g Sucrose was added to 1000 ml water.
Bovine serum albumin: purchased from Sigma-Aldrich (St. Louis, Mo., USA), product code: P 0914.
Butyl acetate: purchased from Nanjing Chemical Reagent Co., Ltd, P. R. China, product code: 1370401001.
Cyclohexane: purchased from Nanjing Chemical Reagent Co., Ltd, P. R. China, product code: 1370400701.
Sodium hydroxide: purchased from Nanjing Chemical Reagent Co., Ltd, P. R. China, product code: 1370500701.
Hydrochloric acid: purchased from Nanjing Chemical Reagent Co., Ltd, P. R. China, product code: 1370500101.
Sodium dihydrogen phosphate: purchased from Nanjing Chemical Reagent Co., Ltd, P. R. China, product code: 1370503001.
Disodium hydrogen phosphate: purchased from Nanjing Chemical Reagent Co., Ltd, P. R. China, product code: 1370503101.
WH-3 Mini vortex mixer: Shanghai Qingpu Huxi Instrument Factory, P. R. China.
XHF-1 High speed dispersing instrument: Shanghai Kingdom Biochemical Instrument Co., Ltd, P. R. China.
U-3000 Spectrophotometer: Hitachi, Ltd., Tokyo, Japan.
Eppendorf centrifuge: 5415 R, German.
HH-S electric-heated thermostatic water bath: Jangsu Dongtai Electric Instrument Factory, P. R. China.
GC-1200r Radioimmunity counter: Science & Technology Industrial General Co., USTC (USTCTEK), P. R. China.

3. Administration and Doses
Non CMS-treated Saline group: 10 ml/kg/day.
CMS-treated Saline group: 10 ml/kg/day.
Composition I (200) group: 200 mg/kg/day.
Composition I (150) group: 150 mg/kg/day.
Fluoxetine group (positive control): 10 mg/kg/day.

The above substances at various concentrations were dissolved in saline. The volume of the suspension administered was based on body weight measured immediately prior to each dose, respectively. All groups were administered orally for six weeks, once per day. All groups except for Non-CMS-treated saline group received CMS treatment as described below.

Method

Sucrose Intake Tests (Measuring the Sucrose Levels in Rats)

All animals were trained to consume 1% (w/v) sucrose solution in water. The training was conducted twice a week for consecutive 3 weeks. In the training, the rats in the home cage were deprived of food and water for 14 hours. After 14 hours, about 30 ml of 1% (w/v) sucrose solution in a pre-weighed bottled was placed in the home cages. 1 hour later, the bottle was taken out from the cage and weighed. The sucrose intake was calculated as follows:

Calculation: Weight of Bottle Before Intake (g)—Weight of Bottle After Intake (g)

After three weeks of training, when the sucrose intake of rats was stabilized, the rats started to receive further treatment. During the whole treatment period, the sucrose intakes of rats were monitored once every week from 9:00 a.m. to 10:00 a.m. with the same procedures as described above.

CMS Treatment

After the three-weeks training, the rats were randomly divided into two groups: the non-CMS treated group and CMS-treated group. The stress group of animals was subjected to the CMS procedure for a period of four consecutive weeks. Each week of stress regime is consisted of six different stress situations. They involved two periods of food and water deprivation, two periods of 45 o cage tilt, two periods of intermittent illumination (lights on and off every 2 h), two periods of soiled cage (200 mL water in sawdust bedding), two periods of paired housing, and two periods of low intensity stroboscopic illumination (150 flashes/min). All of the stressors were 12-14 h of duration and were applied individually and sequentially, day and night.

Each animal in the non-CMS treated group was also housed individually in a home cage under identical conditions. They had free access to food and water in their home cage during the experiment, except for the 14 hours of food and water deprivation before the sucrose intake test conducted once every week.

Drug Treatment

After the animals in the CMS-treated group received CMS treatment for 4 weeks, their sucrose consumption was significantly reduced. Animals therein were divided randomly into the following four subgroups:
(1) Composition I (200) group: received composition I at 200 mg/kg/day;
(2) Composition I (150) group: received composition I at 150 mg/kg/day;
(3) Fluoxetine group: received fluoxetine 10 mg/kg/day; (positive control);
(4) CMS-treated saline group: received saline 10 mL/kg/day. (negative control).

The rats in non-CMS treated group received saline solution at 10 mL/kg orally. All administrations were given orally once daily at 10:00 a.m. for consecutive 6 weeks.

Blood Sampling and Tissue Collection

24 h after the last test of sucrose intake, between 10:00 a.m. and 11:00 a.m., all animals were decapitated quickly to get venous blood. Serum was separated by centrifugation at 3000 rpm and stored at −20° C. until assay of cortisol concentration. Brain tissues were rapidly removed on the ice-plate. The tissues were washed with cold saline, blotted dry and stored at −80° C. until assays of monoamine oxidase A and B (MAO-A and MAO-B) activity.

MAO Assay

Rat brain mitochondrial fractions were prepared following the procedure described in Schurr and Livne, 1976. MAO activity was assessed by spectrophotometer as described in Yu et al., 2002. Briefly, the mitochondrial fraction suspended in 9 vol. of cold sodium phosphate buffer (10 mM, pH 7.4, containing 320 mM sucrose), was mingled at 4° C. for 20 min. at 4° C. for 20 min. The mixture was centrifuged at 4000 rpm for 10 min at 4° C. and the supernatant was re-centrifuged at 15000×g for 30 min at 4° C. to get the protein deposition. Then the deposition was re-suspended in the same buffer. The protein concentration was adjusted to 1 mg/ml. Protein concentration was estimated by the Lowry method (Lowry et al., 1951) using bovine serum albumin as the standard. The assay mixtures contained 4 mM 5-HT or 2 mM β-PEA as specific substrates for MAO-A and MAO-B, respectively, 200 μl solution of the mitochondrial fraction. 10 mM sodium phosphate buffer (pH 7.4) was added to the assay mixture to a final volume of 1 ml. The reaction was allowed to proceed at 37° C. for 20 min, and stopped with 200 μl of 1 M HCl. The reaction product was extracted into 4 ml of butylacetate (for MAO-A assay) or 4 ml of cyclohexane (for MAO-B assay), respectively. The organic phase was measured at wavelength of 280 nm and 242 nm for MAO-A and MAO-B assay with spectrophotometer, respectively. Blank samples were prepared by adding 200 μl of 1 M HCl prior to reaction, and worked up subsequently in the same manner.

Cortisol Assay

Serum cortisol level was assayed using a radioimmunoassay method following the manufacturer's instructions. (Manufacturer: Bejing Furui Biotechnology Company, P.R. China. Product code: FR-FJ-055).

Statistical Analysis

All data were expressed as mean SEM, and analyzed using one-way analysis of variance (ANOVA). A value of p<0.05 was considered to be statistically significant in all the cases. The calculation of P values was performed by employing the Bonferroni alpha correction to avoid false positive results.

Results

Effects on Sucrose Intake

Table 5 shows the effects of drug treatment on sucrose intake (g) in CMS-treated rats (Mean±S.E.M., n=8). It can be seen that none of the consumption of 1% sucrose solution significantly changed in non-CMS treated animals during this experiment. On the other hand, the CMS-treated group showed a decrease in sucrose intake. Before the start of the drug treatment, sucrose intakes in non-CMS and CMS-treated animals were significantly different. Animals in the negative control subgroup that received CMS treatment showed significant and sustained reduction of sucrose intake during the experiment.

Compared to the sucrose intake at week 0, treatment with Composition I and fluoxetine on CMS rats caused a gradual recovery of the sucrose intake. With the treatment of Composition I at the dose of 200 mg/kg, the CMS rats showed significant increase in the sucrose intake compared with week 0, and the sucrose intake was almost recovered to normal after three-weeks. With the treatment of Composition I at the dose of 150 mg/kg, the sucrose intake was significantly elevated and returned to normal after five weeks. On the other hand, the sucrose intake recovery among fluoxetine-treated rats was not as strong as those treated with Composition (I).

TABLE 5

| | Duration of drug treatment (weeks) | | | | | | |
|---|---|---|---|---|---|---|---|
| Groups | 0 | 1 | 2 | 3 | 4 | 5 | 6 |
| Non-CMS treated rats | 13.4 ± 1.3 | 13.5 ± 1.6 | 13.8 ± 1.6 | 14.1 ± 1.9 | 13.8 ± 1.1 | 13.4 ± 1.9 | 14.6 ± 1.7 |

CMS-treated rats

| | | | | | | | |
|---|---|---|---|---|---|---|---|
| Negative control | 5.7 ± 1.2** | 6.2 ± 1.3* | 5.1 ± 1.3** | 6.8 ± 1.6** | 7.1 ± 1.7** | 6.6 ± 0.8** | 7.3 ± 1.3** |
| Composition I (200 mg/kg) | 5.8 ± 1.8** | 8.5 ± 1.8** | 9.5 ± 1.4^{##} | 8.8 ± 1.4**^{#} | 12.6 ± 1.7^{##} | 12.3 ± 1.3^{##} | 15.6 ± 1.5^{##} |
| Composition I (150 mg/kg) | 6.0 ± 1.3** | 7.3 ± 1.0** | 7.4 ± 1.5** | 8.6 ± 1.3**^{#} | 9.3 ± 2.0*^{#} | 10.5 ± 1.6^{##} | 12.7 ± 1.9^{##} |
| Fluoxetine (10 mg/kg) | 5.3 ± 1.4** | 5.5 ± 1.3** | 5.9 ± 1.4* | 7.3 ± 1.5** | 8.9 ± 1.1**^{#} | 10.4 ± 1.3*^{##} | 13.9 ± 1.3^{##} |
| (positive control) | | | | | | | |

Statistical significance:
*P < 0.01,
**P < 0.001 as compared to negative control group at each time-point.
^{#}P < 0.01,
^{##}P < 0.001 as compared to drug-treated stressed animals at Week 0

Effects on Brain MA O-A and MA O-B Activities

Table 6 shows the effects of drug treatment on brain MAO-A and MAO-B activities in CMS treated rats (Mean±S.E.M., n=8). It can be seen that CMS-treated animals resulted in significant increases of cerebral MAO-A and MAO-B activities compared to the negative control group. Oral administration of Composition I at the doses of 200 and 150 mg/kg significantly inhibited MAO-A and MAO-B activities: 45.49% and 12.88% inhibition of MAO-A activity and 64.01% and 46.28% inhibition of MAO-B activity, respectively. Fluoxetine at 10 mg/kg also depressed the enzyme activities, providing 16.43% inhibition for MAO-A activity, 54.05% for MAO-B activity.

TABLE 6

| | | | | MAO | |
|---|---|---|---|---|---|
| | | | | inhibition | |
| | Dose | MAO activity (U/g protein) | | (%) | |
| Groups | (mg/kg) | A | B | A | B |
| Non-CMS treated rats | — | 67.59 ± 6.02 | 63.32 ± 6.82 | | |

CMS-treated rats

| | | | | | |
|---|---|---|---|---|---|
| Negative control | | 93.40 ± 4.82^{##} | 118.60 ± 9.56^{##} | | |
| Composition I | 200 | 50.91 ± 4.05**^{#} | 42.68 ± 3.03***^{#} | 45.49 | 64.01 |
| | 150 | 81.37 ± 5.62 | 63.71 ± 7.70* | 12.88 | 46.28 |
| Fluoxetine | 10 | 78.05 ± 3.68 | 54.50 ± 3.56***^{#} | 16.43 | 54.05 |
| (positive control) | | | | | |

Statistical significance:
^{#}P < 0.05,
^{##}P < 0.01 vs. normal rats,
*P < 0.05,
**P < 0.01,
***P < 0.001 vs. CMS-treated control rats.

Effects on Serum Cortisol Levels

Table 7 shows the effects of Composition I on serum cortisol levels in rats exposed to CMS (Mean±S.E.M., n=8). Serum cortisol levels increased significantly in the CMS-treated rats when compared to the Non-CMS treated group. CMS-treated animals that received Composition I showed significant decrease of serum cortisol levels compared with CMS-treated animals that received saline. Composition I when given at 200 mg/kg could normalize the cortisol levels. On the other hand, administering fluoxetine to CMS-treated animals did not cause any significant alteration of the serum cortisol levels in the present study.

TABLE 7

| Group | Dose (mg/kg) | Cortisol level (ng/ml) |
|---|---|---|
| Normal rats | — | 18.46 ± 0.53 |
| CMS-treated rats | | |
| Control | — | 32.5 ± 0.41^{#} |
| Composition I | 200 | 16.24 ± 0.38** |
| | 150 | 22.62 ± 0.44* |
| Fluoxetine | 10 | 28.76 ± 0.56 |

Statistical significance:
^{#}P < 0.01 vs. normal rats,
*P < 0.05,
**P < 0.01 vs. CMS-treated control rats.

Conclusion

1. The compositions of Buguzhi and Fuling extracts at proper ratios and doses are effective on recovering the sucrose intake in CMS-treated rats, having statistically significant difference from the saline control group.

2. The compositions of Buguzhi and Fuling extracts at proper ratio and doses are effective on inhibiting the MAO-A and MAO-B activities in CMS-treated rats, having statistically significant difference from the saline control group.

3. The compositions of Buguzhi and Fuling extracts at proper ratios and doses are effective on lowering the cortisol level in CMS-treated rats, having statistically significant difference from the saline control group.

4. Oral Administration of Composition I produced beneficial effects on the CMS-treated rats by improving the behavior of the rats in terms of sucrose consumption and exhibited to possess potent and rapid antidepressant properties. The reversal of CMS-induced behavioral change by Composition I is the result of MAO activity inhibition and normalization of the hypothalamic-pituitary-adrenal (HPA) axis hyperactivity. The antidepressant actions could make Composition I of Buguzhi and Fuling extracts a potentially valuable drug for the treatment of elderly depression.

Although the extraction of Buguzhi is described in Example 1, there can be other ways to extract Buguzhi, for example, the air-dried seeds of P. corylifolia (100 g) were powdered. The powder was placed in a 2500 ml round-bottom flask. 800 ml of 65% ethanol was added to the flask, forming the Buguzhi solution. The flask and its contents were immersed into a water bath for 1.5 hours at 85-90° C. before pouring the Buguzhi solution into the percolator for filtering. The filtrate was put in the round-bottom flask again to repeat the same ethanol adding and water bath procedure. The filtrate was concentrated at 60° C. to obtaining 250.0 mL mixture liquid in a rotavapor apparatus (Buchi Labortechnik AG, Switzerland). The mixture liquid was extracted for 8 times, each time using 250 ml of 100% ethyl acetate. The ethyl acetate phase was collected, and the solvent was evaporated under reduced pressure at 60° C. until most of ethyl acetate had been removed. The residue from the ethyl acetate extraction was dried under reduced pressure at 60° C., obtaining 12.21 g of Buguzhi extract (yield 12.21%).

The preparation of the compounds of the present invention is illustrated in the above schemes. While the above examples illustrate the preparation of certain specific embodiments of the present invention, those skilled in the art would be able to prepare the full range of the claimed compounds using methods analogous to those illustrated above.

Also, unless otherwise defined, all technical and scientific terms used herein have the same meaning as commonly understood by one of ordinary skill in the art to which this invention belongs. Although many methods or materials similar to those described herein can be used in the practice or testing of the present invention, only the preferred embodiments are described. Utilizing the description below, a person skilled in the art of the preparation and use of Chinese herbal medicine can readily practice the methods of the present invention.

While dried material is traditionally used and preferred in Chinese herbal medicine, it must be recognized that drying of plant materials facilitates their storage, transportation and subsequent processing. Drying may not be a requirement to derive the benefits of these herbs. As such, it is understood that the present invention may be practiced with the corresponding quantity of the listed fresh plant materials as well. The use of fresh plant materials, sufficient to meet the requisite quantity and proportions of the extracts used, come under the scope of the present invention.

In addition, it is recognized that certain plant parts may contain the active components of interest in higher concentration and the present invention teaches the use of specific plant parts under the standardized nomenclature of the Pharmacopoeia of the People's Republic of China. However these components may also be present in the other parts of the same plants. As such, the components of interest may also be extracted from other parts of the same plant under the scope of the present invention. It is understood that several species within a plant genus may be given under a single plant entry in the Pharmacopoeia and these species with the same genus may be freely substituted by, or used in conjunction with, other members of the same genus as given in the Pharmacopoeia.

A person skilled in the art will appreciate that it is possible, with plant cell and tissue culture techniques, to culture the cells and tissue of these herbs in vitro and to extract the active components of interest from these cells and tissue. Thus, while the extraction of these active components from dried plant parts is preferable and taught, the extraction of these components from plant cells and tissue in culture remain within the scope and spirit of the present invention.

The generalized extraction process comprises reducing the size of the herbal materials followed by extraction by refluxing with a suitable extractant. The herbal extract may be obtained in the following manner, for example. Firstly, the whole plant, leaves, stems, roots, sclerotium, mycelia, and/or seeds of Buguzhi or Fuling are soaked in an extractant, or refluxed with the extractant. There is no limitation in the type of the extractant used. Examples of the extractant which may be used are organic solvents such as methanol, ethanol, propanol, butanol, propylene glycol, 1,3-butylene glycol, glycerin, acetone, methyl ethyl ketone, ethyl acetate, ethers, chloroform and dichloromethane as well as water. These solvents may be used alone or in combination with one another. In the present invention, it is preferable to use methanol, ethanol, ethyl acetate or a mixture of these solvents with water. More preferably, ethanol or a mixture of water and ethanol is used, in the light of the safety (low toxicity) in the living body.

Here, the reducing in size may be achieved by a number of ways including, but not limited to, cutting, chopping, mincing, pounding, pulverizing, milling and grinding. While one way may be taught, other ways and means of achieving a reduction in size of the materials may also be used. As such, these methods and materials fall within the scope of the appended claims.

While an example of how the present invention may be practiced is taught above, a person skilled in the art will also recognize that it is for illustration only and that many equivalent and alternative steps are possible in the preparation method without departing from the scope and spirit of the invention.

Variations may nevertheless occur depending upon the species of mammal, fish or bird being treated and its individual response to said medicament, as well as on the type of pharmaceutical formulation chosen and the time period and interval at which such administration is carried out. In some instances, dosage levels below the lower limit of the aforesaid range may be more than adequate, while in other cases still larger doses may be employed without causing any harmful side effects, provided that such larger doses are first divided into several small doses for administration throughout the day.

The active compounds may be administered alone or in combination with pharmaceutically acceptable carriers or diluents by the routes previously indicated, and such administration may be carried out in single or multiple doses. More particularly, the active compounds may be administered in a wide variety of different dosage forms, i.e., they may be combined with various pharmaceutically acceptable inert carriers in the form of tablets, capsules, lozenges, troches, hard candies, powders, sprays, creams, salves, suppositories, jellies, gels, pastes, lotions, ointments, aqueous suspensions, injectable solutions, elixirs, syrups, and the like.

Such carriers include solid diluents or fillers, sterile aqueous media and various non-toxic organic solvents, etc. Moreover, oral pharmaceutical compositions can be suitably sweetened and/or flavored. In general, the active compounds are present in such dosage forms at concentration levels ranging from about 5.0% to about 70% by weight.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols.

When aqueous suspensions and/or elixirs are desired for oral administration, the active compound may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of an active compound in either sesame or peanut oil or in aqueous propylene glycol may be employed. The aqueous solutions should be suitably buffered (preferably pH greater than 8) if necessary and the liquid diluent first rendered isotonic. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques will known to those skilled in the art.

Additionally, it is also possible to administer the active compounds of the present invention topically and this may be done by way of creams, jellies, gels, pastes, patches, ointments and the like, in accordance with standard pharmaceutical practice.

For administration to animals other than humans, such as cattle or domestic animals, the active compounds may be administered in the feed of the animals or orally as a drench composition.

The active compounds may also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The disclosures of all references cited herein, including those listed below, are incorporated herein by reference in their entireties.

### REFERENCES

Dubois, M., Gilles, K. A., Hamilton, J. K., Rebers, P. A., Smith, F., 1956. Colorimetric method for determination of sugars and related substances. Anal. Chem. 28, 350-356.

Lowry, O. H., Rosebrough, N. J., Farr, A. L., Randall, R. J., 1951. Protein measurement with folin phenol reagent. J. Biol. Chem. 193, 265-275.

Porsolt, R. D., Bertin, A., Jalfre, M., 1977. Behavioural despair in mice: a primary screening test for antidepressant. Archives Internationales de Pharmacodynamie et de Therapie 229, 327-336.

Schurr, A., Livne, A., 1976. Different inhibition of mitochondrial monoamine oxidase from brain by hashish components. Biochemical Pharmacology 25, 1201-1203.

Steru, L, Chermat, R, Thierry, B, Simon, P. 1985. The tail suspension test: a new method for screening antidepressants in mice. Psychopharmacology (Berl) 85:367-370.

Yu, Z. F., Kong, L. D. Chen, Y., 2002. Antidepressant activity of aqueous extracts of Curcuma longa. Journal of Ethnopharmacology 83, 161-165.

Willner, P. 1997. Validity, reliability and utility of the chronic mild stress model of depression: a 10-year review and evaluation. Psychopharmacology (Berl) 134, 319-29.

What is claimed is:

1. A pharmaceutical composition comprising the extract of Psoralea corylifolia L. and extract of Poria cocos (Schw.) Wolf., wherein said composition is in the absence of the herbs selected from the group consisting of Radix Ginsen, Radix Polygoni Multiflori, Radix Ophiopogonis, Radix Codonopsis, Radix Astragali, Rhizoma Atractylodis Macrocephalae, Fructus Ligustri Lucidi, Radix Salviae Miltiorrhizae, Rhizoma Paridis, Rhizoma Cyperi, Rhizoma Atractylodis, Radix Saposhnikoviae, Herba Ecliptae, Fructus Tribuli, Radix Lithospermi, and Radix Glycyrrhizae.

2. A pharmaceutical composition according to claim 1, consisting essentially of the extract of Psoralea corylifolia L. and the extract of Poria cocos (Schw.) Wolf.

3. A pharmaceutical composition according to claim 1, consisting of the extract of Psoralea corylifolia L. and the extract of Poria cocos (Schw.) Wolf.

4. The pharmaceutical composition according to claim 1, wherein said extract of Psoralea corylifolia L. is Buguzhi extract and said extract from Poria cocos (Schw.) Wolf. is Fuling extract.

5. The pharmaceutical composition according to claim 4, wherein the weight portion of Buguzhi extract and Fuling extract ranges from about 1:1 to about 1:7.

6. The pharmaceutical composition according to claim 4, wherein the portion of Buguzhi extract and Fuling extract ranges from about 1:1 to about 1:3.

7. The pharmaceutical composition according to claim 4, wherein the portions of Buguzhi extract and Fuling extract is about 1:1.5.

8. The pharmaceutical composition according to claim 4, wherein the Buguzhi extract comprises about 95% furocoumarins, wherein said furocoumarins comprising about 7-9% of Psoralen and about 6-8% of Isopsoralen.

9. The pharmaceutical composition according to claim 4, wherein the Fuling extract comprises about 57-62% Fuling Polysaccharides.

10. A pharmaceutical composition comprising furocoumarins and Fuling polysaccharides, wherein said furocoumarins comprising Psoralen and Isopsoralen.

11. The pharmaceutical composition according to claim 10, wherein the proportions of psoralen, isopsoralen and Fuling polysaccharides in the composition is 7-9:6-8:86-93.

12. A method of preparing the pharmaceutical composition of claim 4, comprising:
a. extracting Buguzhi to form Buguzhi extract;
b. extracting Fuling to form Fuling extract, and
c. combining said Buguzhi extract and said Fuling extract.

13. The method according to claim 12, wherein the weight ratio of Buguzhi to Fuling ranges from about 1:2 to about 2:1.

14. The method according to claim 12, wherein the weight portion of Buguzhi to Fuling ranges from about 1:1 to about 2:1.

15. The method according to claim 12, wherein the weight portion of Buguzhi to Fuling is about 2:1.

16. The method according to claim 12, wherein said extracting of Buguzhi in step a. further comprises:
i. extracting said Buguzhi with a first extractant to form a first extract solution;
ii. extracting said first extract solution with a second extractant to form a second extract solution comprising residue; and
iii. isolating and drying said residue from said second extract solution;
wherein one of said extractants is a high-polar solvent, the other of the extractants is a low-polar solvent.

17. The method according to claim 16, wherein said first extractant is selected from the group consisting of ethanol, methanol, and acetone.

18. The method according to claim 16, wherein said first extractant is 65% (v/v) ethanol.

19. The method according to claim 16, wherein said second extractant is selected from the group consisting of ethyl acetate, ether, chloroform, and dichloromethane.

20. The method according to claim 16, wherein said second extractant is 100% (v/v) ethyl acetate.

21. The method according to claim 12, wherein the extracting of Fuling in step b. further comprises
i. extracting Fuling with water to obtain a water-soluble extract and a water-insoluble Fuling residue;
ii. extracting the water-insoluble Fuling residue with an alkaline solvent to obtain an alkali-soluble extract; and
iii. combining said water-soluble extract and alkali-soluble extract to form said Fuling extract.

22. The method according to claim 21, wherein step (i) further comprises
a. decocting the Fuling in water to form a Fuling solution;
b. filtering and concentrating said Fuling solution to form a concentrated Fuling solution;
c. precipitating said concentrated Fuling solution to form precipitation;
and
d. isolating and drying said precipitation to obtain the water-soluble extract.

23. The method according to claim 22, wherein said precipitation in step (c) is formed by adding alcohol.

24. The method according to claim 23, wherein said alcohol is 95% (v/v) ethanol.

25. The method according to claim 21, wherein step (ii) further comprises:
a. extracting the water-insoluble Fuling residue with an alkaline solvent to form an extract solution, wherein said extract solution having a pH value of 11-13;
b. neutralizing said extract solution with acid to pH=6-7 to form a neutralized extract solution;
c. precipitating said neutralized extract solution to form precipitation;
and
d. isolating and drying said precipitate to obtain the alkali-soluble extract.

26. The method according to claim 25, wherein said alkaline solvent is 1M NaOH.

27. The method according to claim 25, wherein said precipitation is formed by adding alcohol.

28. The method according to claim 27, wherein said alcohol is 95% (v/v) ethanol.

29. A method of treatment of depression, comprising administering of a therapeutically effective amount of the pharmaceutical composition of claim 1.

30. The method according to claim 29, wherein the effective amount is 100-250 mg/kg/d.

31. The method according to claim 29, wherein said treatment comprises modulating at least one of the following symptoms: anxiety, insomnia, stress, nervousness, emotional disorder, disability to adapt to pressure, despair, failure to adapt to stress, enhanced cortisol level, and enhanced MAO-A and MAO-B activities in the brain, reduced energy, chronic fatigue syndrome, postpartum depression, anxious depression, atypical depression, anxious mood, irritable mood, and melancholic and atypical symptom, violent acts, suicidal behavior or suicidal ideation, sleep disturbance, decrease in appetite, eating disorder, concentration, psychomotor agitation/retardation.

32. A health supplement comprising the pharmaceutical composition of claim 1.

33. A nutritional supplement comprising the pharmaceutical composition of claim 1.

34. A formulation for treating depression comprising composition of claim 4, wherein the concentration of said composition ranges from 1% to 100% by weight.

35. The formulation according to claim 34, wherein the ratio of Buguzhi extracts to Fuling extract ranges from 1:1 to 1:7.

36. The formulation according to claim 34, wherein the ratio of Buguzhi extracts to Fuling extract ranges from 1:1.5 to 1:6.

37. The formulation according to claim 34, wherein the ratio of Buguzhi extracts to Fuling extract is from 1:1.5 to 1:3.
